(19) United States
(12) Patent Application Publication
(10) Pub. No.: US 2012/0252100 A1
(43) Pub. Date: Oct. 4, 2012

(54) Mixed Strain Culture For The Disposal Of Food Waste, And Food Waste Disposal Method Using Same

(76) Inventors: You Jung Jung, Wonju-si (KR); Tae Seok Ahn, Seoul (KR); Da Woon Jung, Chuncheon-si (KR); Ahn Na Cho, Chuncheon-si (KR); Eun Young Lee, Seoul (KR); Myoung Sun Lee, Seoul (KR)

(21) Appl. No.: 13/518,723

(22) PCT Filed: Dec. 23, 2010
(86) PCT No.: PCT/KR10/09274

§ 371 Date: Jun. 22, 2012

(30) Foreign Application Priority Data
Dec. 24, 2009 (KR) 10-2009-0131129

(51) Int. Cl. C12N 1/20; C12S 3/00

(52) U.S. Cl. 435/252.4; 435/267

(57) ABSTRACT

The present invention relates to a mixed strain culture for the disposal of food waste, and more particularly, to a mixed strain culture for the disposal of food waste which has high degradation activity on cellulose, amylose, protein, and fat at a wide range of temperatures, pH levels, and salinities, and which can degrade food waste having a high moisture content and therefore can degrade food waste in an efficient manner. The present invention also relates to a food waste disposal method using the mixed strain culture.

### TECHNICAL FIELD

The present invention relates to a mixed strain for the disposal of food waste. More specifically, the present invention relates to a mixed strain for the disposal of food waste which has high degradation activity on cellulose, amylose, protein and fat in a wide range of temperature, pH and salinity, and can degrade food waste having high moisture content, and therefore can degrade food waste in an efficient manner, and a method for the disposal of food waste using the same.

### BACKGROUND ART

In the Republic of Korea, the amount of domestic waste generated is 50,346 tons per day (in 2007), and the generation of waste has been sharply increasing over the last twenty (20) years due to population growth, industrialization of cities and urban concentration. With respect to the disposal of waste, in 1991 89.2% of domestic waste was buried and only 7.9% was recycled. However, in 2000 the ratio of landfill was decreased to 47.0% and recycling was increased to 41.3% due to a volume-rate garbage disposal system and recycling policies.

Among them, food waste has been mainly disposed by burying it in a landfill. However, when food waste is buried in a landfill, it can be a direct cause of leachate due to high moisture content, and food waste is easily decomposed in the course of the collection and transportation, and thus various problems such as stench may be caused. As a result, the direct use of landfill for food waste has been prohibited since 2005. Because of the prohibition of the use of landfill, the separate collection of food waste has been established. However, due to population growth, the improvement of income and the change of lifestyle, food waste generated an average of 11,577 tons per day in 1999 had increased to 14,452 tons by 2007.

Another disposal method is incineration, but the deterioration of incineration efficacy due to high moisture content makes the cost of disposal high, and environmentally toxic substances such as dioxin may be generated. To address such problems, recycling methods of food waste such as compost, feed or energy production therefrom have been begun in earnest.

Anaerobic digestion has advantages over compost production in that the subject matter is a wet biodegradable waste and energy can be retrieved, but it also has disadvantages in that large facilities are needed and the cost of maintenance is high. As methods of producing compost, decomposing type, fermentation type and drying type have been representatively installed and operated. The decomposing type in which a bulking agent (sawdust, chaff, coco peat, etc.) and microbes are added, and then food waste (organic material) is decomposed—has problems in the imbalance between supply and demand, and the rising price of bulking agent. In the case of drying type, food waste is incorporated and heated by hot wind, a heater or an indirect steam heater to 70-120° C. to evaporate moisture, and the dried content is crushed by agitation and its weight is reduced. However, the drying type is ineffective in a practical aspect in view of the economics of heating apparatus. High-temperature aerobic type, which uses thermal energy generated by a microbe reaction without an external additional heat supply, has been studied as one of the disposal methods of organic wastewater. However, due to the lack of various control techniques, it has not yet been practically applied. The activation of thermophilic microbes is crucial in this disposal method, but there is no method for maintaining temperature consistently so it has not yet been commercialized as other currently used wastewater disposal methods.

The advantages of such methods using reaction heat are that the disposal rate of organic materials is rapid since a reaction is carried out at a high temperature, stable disposal efficacy over organic materials load can be shown, efficient use of supplied oxygen can be efficiently used since no nitrification occurs at a high temperature, and stable resultants of disposal can be obtained.

In the case of compost production type and decomposing type, the need for a bulking agent such as sawdust makes the volume of apparatus large, and thus the cost for waste disposal is increased. In addition, in the course of making compost, most organic materials are converted into carbon dioxide, ammonia, mercaptan generated in aerobic and anaerobic states, water, microbe cells, thermal energy and humus. Among them, because ammonia and mercaptan generate pungent smells, it is inconvenient and costly to use deodorizer and platinum catalyst to remove them. Recently, a method of recycling exhaust gas has been employed to reduce such additional costs, but fundamental cost reduction has not yet been accomplished since platinum catalyst for removal of the pungent smells is generally still used.

At present, representative microbes used in the compost production method and decomposing method for the disposal of food waste are those known to be able to decompose cellulose such as coryneform, nocardioform, true filamentous bacteria and actinomycetes. Such microbes play an important role in decomposing hydrocarbon, remnants of plants and soil compost. Some microbes belonging to such groups also decompose insecticide. Mainly filamentous actinomycetes belonging to Streptomyces produce odorous compounds such as geosmin which has a distinct earthy aroma (Parker, 2001). In connection with disposal of organic materials, soil microbes are classified based on (1) preference for substrate which can be used with ease or difficultly, (2) the concentration of substrate needed. At the level of high nutrients, microbes such as Pseudomonas rapidly react to easily available substrates such as sugar or amino acids. Indigenous microbes tend to use natural organic materials to the maximum. Some examples of such microbes are Arthrobacter and many soil actinomycetes. Because actinomycetes rapidly grow at 70% or less of moisture content, in both the existing developed compost production method and decomposing method of waste disposal it is recommend that the moisture content be adjusted between 40 and 60%. However, while such moisture-content regulation is possible in some large-scale waste disposal facilities, it is not possible in most homes, restaurants and markets. This can be identified as the number one cause of failure in efficient waste disposal. In addition, heating to decrease the moisture content may cause large energy consumption, and there is a disadvantage that a sprinkling system for removing degradation products cannot be properly used since low moisture content should be maintained.

Korean Patent No. 0580857 discloses a method for efficient disposal of food waste having high moisture content with high degradation activity by using a mixed strain of Bacillus smithii and thermophilic yeast, ATS-1 (KCTC 10637BP).

However, because food waste shows a wide range of pH and salinity due to its nature, there is a strong demand for developing microbe strains which can more efficiently dispose of food waste in a wide range of temperature, pH and salinity.

### REFERENCE

Parker M. M., Block Biology of Microorganisms (Ninth Edition), 2001, New Jersey: Prentice Hall

### DETAILED DESCRIPTION OF THE INVENTION

### Technical Problem

Therefore, the technical problem of the present invention is to provide a microbe strain which can efficiently dispose of food waste with high degradation activity on cellulose, amylose, protein and fat in a wide range of temperature, pH and salinity.

### Solution to the Problem

To accomplish the above object, the present invention provides a mixed strain (KCTC 11585BP) for the disposal of food waste comprising Brevibacillus borstelensis, Bacillus licheniformis and Kazachstania telluris.

In addition, the present invention provides a method for the disposal of food waste by using said mixed strain.

Hereinafter, the present invention is explained in detail.

The mixed strain for the disposal of food waste according to the present invention comprises Brevibacillus borstelensis, Bacillus licheniformis and Kazachstania telluris, and was deposited at the Korean Collection for Type Cultures (KCTC) under Accession Number KCTC 11585BP on Nov. 10, 2009.

In the present invention, the mixed strain for the disposal of food waste consists of Brevibacillus borstelensis and Bacillus licheniformis which are bacteria, and Kazachstania telluris which is yeast.

Both Brevibacillus borstelensis and Bacillus licheniformis have degradation activity on cellulose, starch, fat and protein, and can survive at salinity as high as 4%. Brevibacillus borstelensis is Gram-positive bacteria and isolated mainly from soil. It is known as a thermophilic microbe producing D-stereospecific amino acid amidase which is an enzyme hydrolyzing amino-terminal amino acid of D-amino acid-containing amide.

Bacillus licheniformis is also Gram-positive bacteria and isolated mainly from soil. It is a thermophilic bacteria capable of growing at high temperature of 50° C. or more and has characteristics of surviving as a spore in unfavorable conditions and growing in favorable conditions.

Kazachstania telluris has an optimal growth temperature of 37 to 45° C., and is capable of degrading and fermenting cellulose, starch and glucose. Kazachstania telluris grows very rapidly, uses nitrate and can ferment various carbohydrates.

There is no literature reporting that Brevibacillus borstelensis, Bacillus licheniformis and Kazachstania telluris can be harmful to health and the environment. In addition, the American Type Culture Collection (ATCC) classified them as biosafety level-1, non-pathogenic bacteria and yeast. Therefore, all strains included in the mixed strain for the disposal of food waste according to the present invention are safe and cause no problems to the human body and the environment.

According to another aspect of the present invention, a method for the disposal of food waste by using the mixed strain (KCTC 11585BP) of the present invention is provided.

The method for the disposal of food waste according to the present invention may be carried out preferably at 30 to 60° C., more preferably 40 to 50° C. Because the mixed strain for the disposal of food waste of the present invention consists of thermophilic bacteria and thermophilic yeast, it can efficiently dispose of food waste at the above high temperature range. The maintenance of decomposing activity at high temperature is one of important factors since the interior temperature of the disposal apparatus increases due to exothermic reaction during the decomposition of food waste. In addition, disposal at high temperature may play a role in maintaining flora by preventing contamination with other microbes as well as in making decomposition of food waste more active.

The mixed strain (KCTC 11585BP) of the present invention may be formulated in various forms for the convenience of transportation or storage. For example, a powder form may be used by freeze drying with a cryoprotectant, and a solid form may be used by mixing the mixed strain with a preservative carrier, adsorbing and drying. There is no specific limitation to the cryoprotectant and preservative carrier, and those conventionally used in the art may be used. For example, glycerol, skim milk, honey and the like may be used as a cryoprotectant, and diatomaceous earth, active carbon, defatted rice bran and the like may be used as a preservative carrier.

### EFFECTS OF THE INVENTION

The mixed strain (KCTC 11585BP) for the disposal of food waste according to the present invention has high degradation activity on cellulose, amylose, protein and fat in a wide range of temperature, pH and salinity, and can degrade food waste having high moisture content. As a result, because the present mixed strain can efficiently degrade various kinds of food waste, it can be disposed at low cost. The yeast included in the mixed strain (KCTC 11585BP) dilutes characteristic odors at the time of degrading food and raises the degradation rate by carrying out alcohol fermentation to help food degradation. Therefore, the present invention can solve the environmental pollution problem caused in the conventional methods for the disposal of food waste such as landfill or incineration and can dispose of food waste in an environmental friendly manner.

### BRIEF DESCRIPTION OF THE DRAWINGS

FIG. 1 is a microscope photograph of Brevibacillus borstelensis with 1,000× magnification.

FIG. 2 is a microscope photograph of Bacillus licheniformis with 1,000× magnification.

FIG. 3 is a microscope photograph of Kazachstania telluris with 1,000× magnification.

FIG. 4 is a graph representing the degradation rate of glucose (5 mg/100 ml) by yeast.

FIG. 5 is a graph representing the degradation rate of glucose (10 mg/100 ml) by yeast.

FIG. 6 is a graph representing the degradation rate of glucose (20 mg/100 ml) by yeast.

FIG. 7 is a graph representing the growth curve of nATS-AG according to temperature.

FIG. 8 is a graph representing the growth curve of Comparative Strain according to temperature.

FIG. 9 is a graph representing the growth curve of nATS-AG according to initial pH.

FIG. 10 is a graph representing the growth curve of Comparative Strain according to initial pH.

FIG. 11 is a graph representing the growth curve of nATS-AG according to salinity.

FIG. 12 is a graph representing the growth curve of Comparative Strain according to salinity.

FIG. 13 is a microscope photograph of carrot with 1,000× magnification 24 hours after strain treatment, and FIG. 14 is a microscope photograph after 48 hours (left: Comparative Strain, right: nATS-AG).

FIG. 15 is a microscope photograph of kelp with 1,000× magnification 24 hours after strain treatment, and FIG. 16 is a microscope photograph after 48 hours (left: Comparative Strain, right: nATS-AG).

FIG. 17 is a microscope photograph of leek with 1,000× magnification 24 hours after strain treatment, and FIG. 18 is a microscope photograph after 48 hours (left: Comparative Strain, right: nATS-AG).

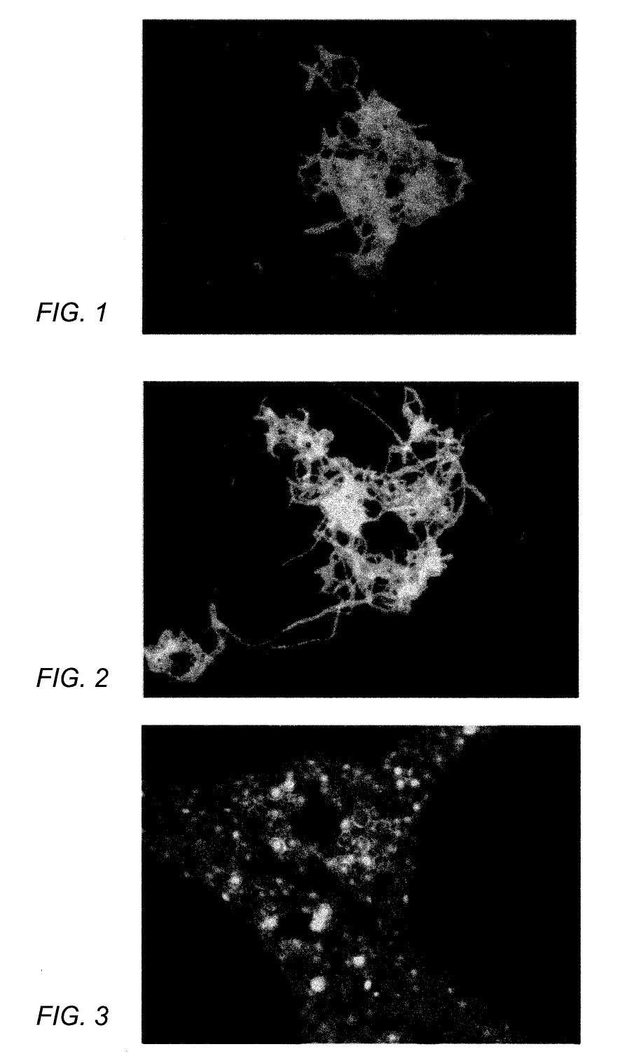

### MODES FOR CARRYING OUT THE INVENTION

The present invention is explained in more detail with the following examples. However, it must be understood that the protection scope of the present invention is not limited to the examples.

### Isolation and Selection of Microbes

To isolate thermophilic strains having excellent capacity to degrade protein, cellulose, starch and fat, samples were collected from a decomposing spot of fallen leaves where organic materials are actively decomposed. Each 1 g of the collected soil and humus samples were added to broth containing 1% carboxymethyl cellulose, 1% starch, 1% peptone and 1% olive oil, and then enrichment culture was carried out at 45° C., 60° C. and 70° C. for 24 hours. The supernatant of cultures was transferred to a solid medium by spread plating. After streaking 3 times, colonies having different size and morphology were selected. The selected colonies were inoculated to nutrient media, incubated for 48 hours, and their growth was measured by OD₆₀₀.

To measure degradation activities of the isolated strains, activity of amylase, cellulase, protease and lipase were measured. Among colonies formed on screening media for cellulase (1% CMC, 1% tryptone, 0.5% yeast extract, 1% NaCl and 1.5% agar), screening media for amylase (0.3% beef extract, 2% soluble starch, 0.5% peptone, 0.5% NaCl and 1.5% agar), screening media for protease (0.5% pancreatic digest of casein, 0.25% yeast extract, 0.1% glucose, 1% skim milk and 1.5% agar) and screening media for lipase (1% Tween80, 1% peptone, 0.5% NaCl, 0.01% CaCl₂H₂O and 1.5% agar), activities were determined by the size of clear zone. After the selection of strains having excellent activities, the two (2) strains that showed the optimal combination were selected and identified by base sequence analysis. In the case of yeast, samples were collected from a mowed lawn disposal site near Chuncheon-si, Gangwon-do and added to YM media (0.3% yeast extract, 0.3% malt extract, 0.5% peptone and 1% dextrose) containing 4 μg/ml of ampicillin for enrichment culture. After confirmation of the existence of yeast by microscopy, the yeast was purely isolated on the same media.

The degradation activity of each strain was evaluated by the following method. The size of ring around colony shown after incubation was measured, and the activity was rated as +++ when ring size was 6 mm or more, ++ when 3 to 5 mm and + when less than 3 mm.

The degradation activity of yeast was evaluated by the consumption rate of reducing sugar. The concentration of reducing sugar was measured by the Somogyi method.

### Identification of Microbes

16S rRNA base sequence analysis of the selected bacteria was carried out. As a result of comparing the result with BLAST of NCBI, the selected bacteria were identified as Brevibacillus borstelensis (98% homology) and Bacillus licheniformis (99% homology). In the case of the yeast, 18S rRNA base sequence analysis was carried out. As a result of comparing the result with BLAST of NCBI, the yeast was identified as Kazachstania telluris (100% homology).

### Evaluation of Degradation Activity

The measured activities for each substrate of the selected bacteria strains are represented in Table 1.

TABLE 1

| Strain | Cellulase | Amylase | Protease | Lipase |
|---|---|---|---|---|
| Brevibacillus | ++ | + | ++ | + |
| borstelensis | | | | |
| Bacillus licheniformis | + | + | ++ | + |

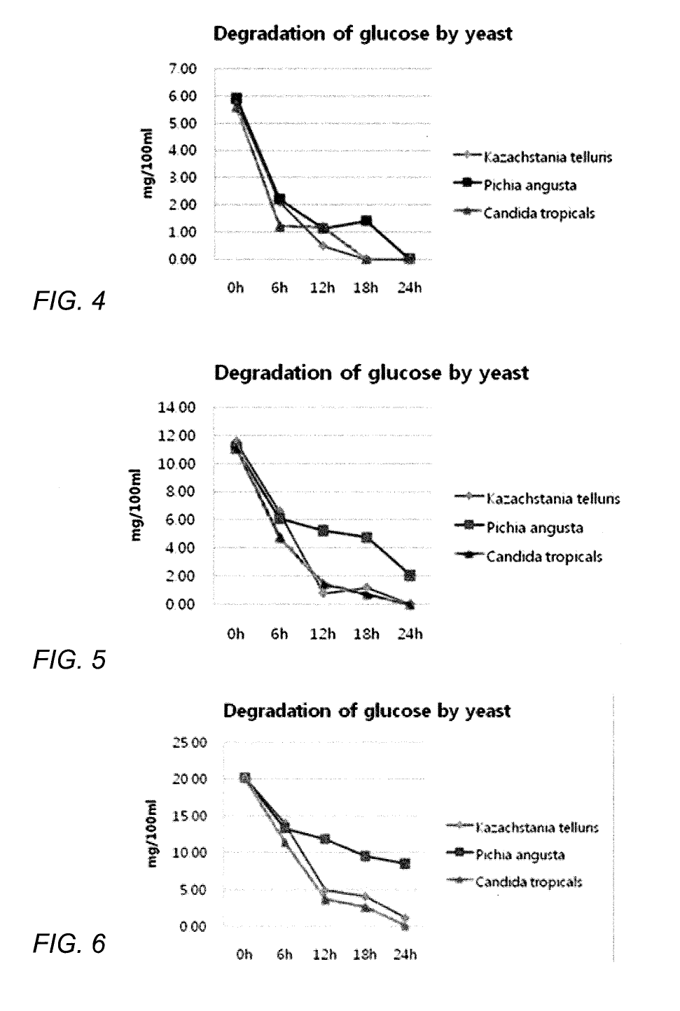

Because yeast plays a role in degrading and absorbing low-molecular weight materials which are degradation products of organic materials, the activity of yeast was measured by the consumption rate of reducing sugar. The concentration of reducing sugar was measured by the Somogyi method. The absorbing and degrading rate of glucose of three (3) yeasts—Kazachstania telluris, thermophilic yeast (Candida tropicals) used in Korean Patent No. 0580857 and thermophilic yeast (Pichia angusta: strain No. 17664) bought from the Korean Collection for Type Cultures (KCTC)—was measured and compared (FIGS. 4 to 6).

As can be seen from FIGS. 4 to 6, the consumption rate of reducing sugar of Kazachstania telluris is similar to that of thermophilic yeast used in Korean Patent No. 0580857 at all concentrations, but that of the strain (Pichia angusta) bought from the KCTC is low.

### Preparation of Mixed Strain

The stocks of Brevibacillus borstelensis and Bacillus licheniformis were added to nutrient broth as 1% concentration and incubated with agitation in a 45° C. incubator for 24 hours. The stock of Kazachstania telluris was added to YM broth as 1% concentration and incubated with agitation in a 37° C. incubator for 24 hours. Each 400 ml of bacteria cultures and 200 ml of yeast culture were mixed to give 1,000 ml of mixed strain.

The obtained mixed strain was deposited at the Korean Collection for Type Cultures (KCTC) under Accession Number KCTC 11585BP on Nov. 10, 2009. (Hereinafter the mixed strain is referred to as “HATS-AG”).

### Experimental Example 1

### Measurement of Growth Rate According to Temperature, Initial pH and Salinity

### Experiment Method

The growth curve of nATS-AG was measured with a variation of temperature, initial pH and salinity. The media consisted of 5 g/l of peptone, 10 g/l of gelatin, 2.5 g/l of yeast extract, 5 g/l of soluble starch, 3 g/l of malt extract, 3 g/l of cellulose, 2 g/l of beef extract and 5 g/l of NaCl. The increase of population is represented by the change of absorbance at 600 nm of a spectrophotometer.

Using the same method as above, the growth curve of the mixed strain (ATS-1) of Bacillus smithii and thermophilic yeast disclosed in Korean Patent No. 0580857 (hereinafter referred to as “Comparative Strain”) was measured and compared with that of nATS-AG.

### Experimental Example 1-1

### Growth Rate According to Temperature

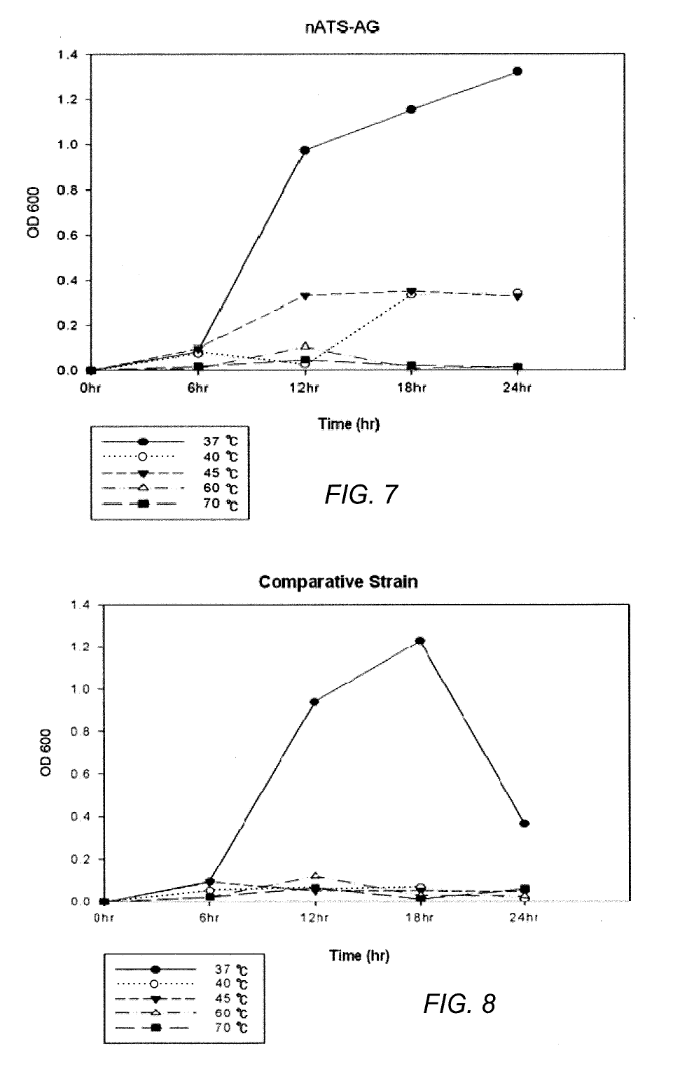

As with the experiment method above, the growth rates of nATS-AG and Comparative Strain at 37° C., 45° C., 60° C. and 70° C. were measured, and the results are represented in FIGS. 7 and 8, respectively.

Contrary to Comparative Strain, nATS-AG sufficiently grows after 24 hours of initiation of incubation and grows even at 45° C. At 37° C., Kazachstania telluris actively grows so that OD value is much higher than other temperatures.

From the above results, it can be known that the growth rate of nATS-AG at high temperature is much higher than that of Comparative Strain.

### Experimental Example 1-2

### Growth Rate According To Initial pH

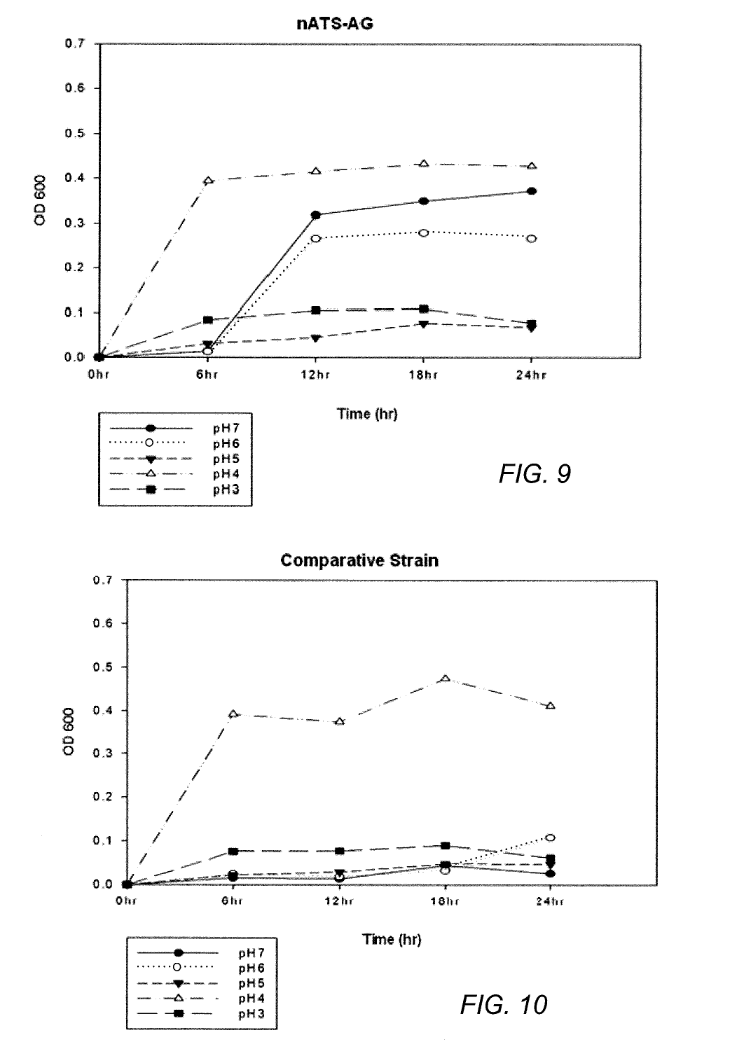

As with the experiment method above, the growth rates of nATS-AG and Comparative Strain at initial pH 3, 4, 5, 6 and 7 were measured, and the results are represented in FIGS. 9 and 10, respectively.

Comparative Strain shows a high growth rate at initial pH 4 only, whereas nATS-AG shows a higher growth rate than Comparative Strain at initial pH 4, 6 and 7. When microbes degrade food, pH is decreased by producing organic acids, but generally not decreased to pH 4 or lower. nATS-AG shows a high growth rate at a various range of initial pH. As a result, it can be known that nATS-AG can be employed helpfully in the disposal of food having various pH and low pH conditions in the course of food degradation.

### Experimental Example 1-3

### Growth Rate According to Salinity

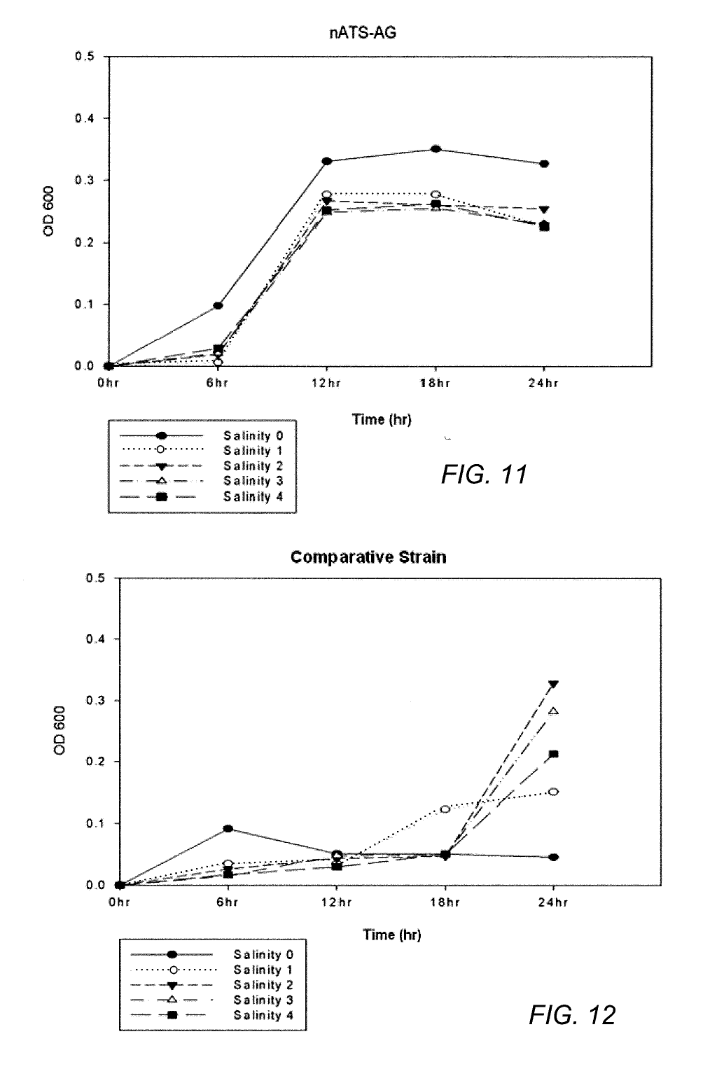

As with the experiment method above, the growth rates of nATS-AG and Comparative Strain at salinity of 0%, 1%, 2%, 3% and 4% were measured, and the results are represented in FIGS. 11 and 12, respectively.

nATS-AG maintains its growth at 4% salinity. Considering that the concentration of salt in Korean food is generally 3% or below and salt is removed by contacting water in the course of disposing food waste, it is believed that there is no problem regarding salinity. In addition, Comparative Strain shows the increase of OD value after 24 hours have elapsed, whereas nATS-AG shows a normal growth curve. Therefore, it can be known that nATS-AG can efficiently degrade food beginning the initial disposal of food as compared with Comparative Strain.

### Experimental Example 2

### Measurement of Degradation Activity

### Experimental Example 2-1

### Small-scale Disposal

The food degradation activity of nATS-AG and Comparative Strain was measured. As a sample, a mixture of rice, lettuce and pork with a mixing ratio of 1:1:1 based on weight was used. Because a food disposal process using microbes is usually carried out by inoculating a strain and continual incorporation of food waste at regular time intervals, it is important that microflora be stably maintained for degradation activity. Therefore, before the experiment, microflora was stabilized for 24 hours. The condition for preparing initial flora is shown in Table 2.

TABLE 2

| Material | Amount |
|---|---|
| Food (rice, lettuce, pork) | 1,200 g |
| Strain (nATS-AG or Comparative | 50 ml |
| Strain) | |

Then, 500 g of food was continually added at an interval of 6 hours. The degradation rate according to each time zone is calculated by the following formula: accumulated total amount (dry weight)−final residual food amount (dry weight)/accumulated total amount (dry weight)×100(%), and its average is calculated. The results are represented in Table 3.

TABLE 3

| | Degradation rate | Degradation rate | Average |
|---|---|---|---|
| | (%/12 hours) | (%/12 hours) | degradation rate |
| Strain | (0-12 hours) | (12-24 hours) | (%) |
| nATS-AG | 49 | 47 | 48 |
| Comparative | 44 | 41 | 42 |
| Strain | | | |

From the results in Table 3, it can be known that nATS-AG can more efficiently dispose of food waste by showing higher degradation activity than Comparative Strain which has been known to have high degradation activity.

### Experimental Example 2-2

### Large-scale Disposal

The experiment to check whether nATS-AG can efficiently degrade a large amount of food waste for a long time was carried out. 1,000 g of food waste collected from the cafeteria of Kangwon National University was incorporated into a disposal apparatus, and each 10 ml of nATS-AG and Comparative Strain were then inoculated. 1,000 g of food waste was additionally incorporated after 12 hours, and then 1,000 g of food waste was additionally incorporated at an interval of 8 hours or 16 hours up to 84 hours. At 20, 44, 68 and 96 hours, prior to the food waste incorporation the weight of food waste remaining after degradation was measured and the weight reduction rate was calculated by the following formula: (1−food waste remaining weight/food waste input weight)×100(%).

The results are represented in Table 4.

TABLE 4

| | Food | Remaining weight (g) | | Weight reduction rate | |
|---|---|---|---|---|---|
| | waste input | Comparative | | Comparative | |
| Hour | weight (g) | Strain | nATS-AG | Strain | nATS-AG |
| 0 | 1,000 | — | — | — | — |
| 12 | 1,000 | — | — | — | — |
| 20 | 1,000 | 720 | 620 | 64.0% | 69.0% |
| 36 | 1,000 | — | — | — | — |
| 44 | 1,000 | 1,420 | 1,070 | 64.5% | 73.3% |
| 60 | 1,000 | — | — | — | — |
| 68 | 1,000 | 1,640 | 1,460 | 72.7% | 75.7% |
| 84 | 1,000 | — | — | — | — |
| 96 | — | 2,890 | 2,500 | 63.9% | 68.8% |

As indicated in Table 4, it can be known that nATS-AG more efficiently degrades food waste and maintains its degradation activity for a long time as compared with Comparative Strain.

### Experimental Example 3

### Degradation Activity for Recalcitrant Food

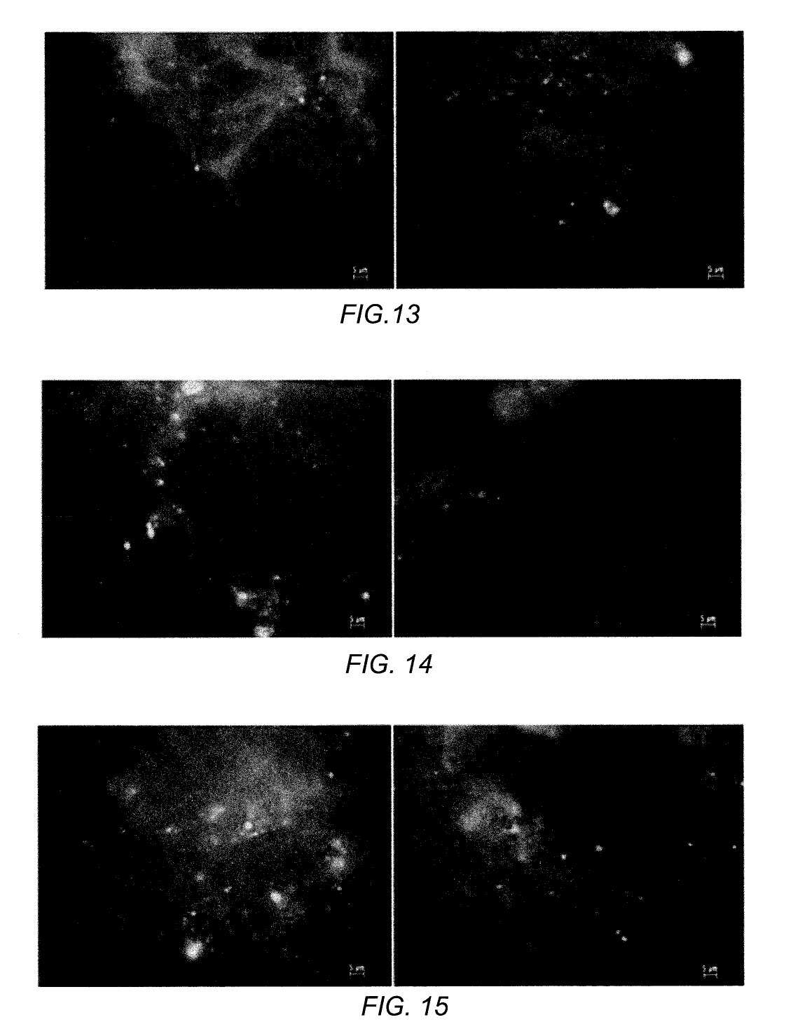

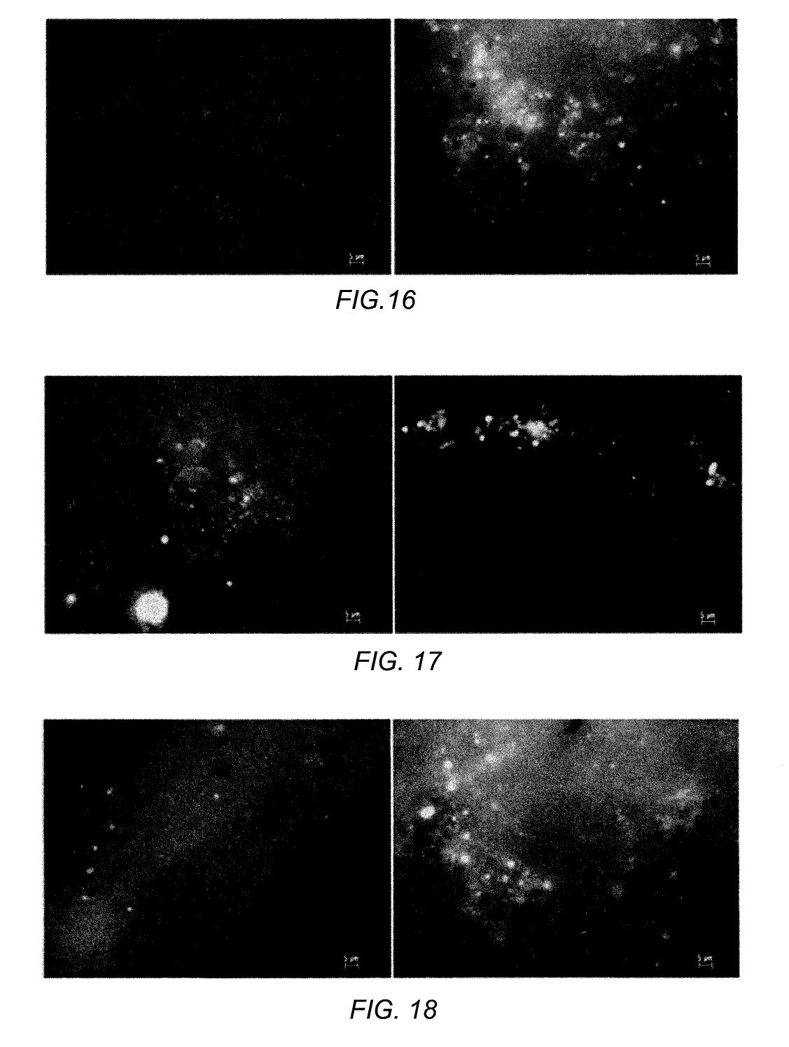

The degradation activity for food known as being difficult to degrade was measured. To increase degradation activity, microbes should adhere well to food and invade tissues well. To evaluate such ability, carrot, kelp and leek were selected as recalcitrant foods. After treatment of nATS-AG and Comparative Strain, food was stained with DAPI and photographed with a fluorescence microscope (BX-60, Olympus) after 24 and 48 hours. The results are shown in FIGS. 13 to 18.

As can be seen from FIGS. 13 to 18, it can be known that nATS-AG more efficiently adheres to food and invade tissues as compared with Comparative Strain.

### Experimental Example 4

### Freeze Drying of Strain and Test of Survival Rate

nATS-AG was inoculated to 5 L of media (0.5% yeast extract, 1% peptone, 2% dextrose, 0.8% nutrient broth and 0.5% malt extract) and incubated. After incubation, 10% (w/v) of skim milk was added thereto as a cryoprotectant and then freeze dried to give 311.26 g of powder.

0.1 g of the obtained freeze-dried strain was suspended in 1×PBS and smeared on nutrient agar and potato dextrose agar media. After 24 hours of incubation in a 37° C. incubator, formed colonies were counted to measure survival rate. The results are represented in Table 5.

TABLE 5

| | | cfu (colony forming |
|---|---|---|
| | Microbe | unit)/g |
| Culture solution | Yeast | 2 × 10⁸ |
| | Bacteria | 8 × 10⁹ |
| Freeze-dried strain | Yeast | 5 × 10⁷ |
| | Bacteria | 3 × 10⁸ |

As can be seen from Table 5, nATS-AG of the present invention maintains a relatively high survival rate after freeze drying.

1. A mixed strain of Brevibacillus borstelensis, Bacillus licheniformis and Kazachstania telluris for the disposal of food waste which is deposited at the Korean Collection for Type Cultures (KCTC) under Accession Number 11585BP.

2. A method for the disposal of food waste by using a mixed strain of Brevibacillus borstelensis, Bacillus licheniformis and Kazachstania telluris deposited at the Korean Collection for Type Cultures (KCTC) under Accession Number 11585BP.

3. The method according to claim 2 wherein the disposal of food waste is carried out at 30 to 60° C.
